(19) United States
(12) Patent Application Publication
(10) Pub. No.: US 2026/0151528 A1
(43) Pub. Date: Jun. 4, 2026

(54) VOLATILE COMPOSITION

(71) Applicant: The Procter & Gamble Company, Cincinnati, OH (US)

(72) Inventors: Rahul VYAS, Singapore (SG); Desmond NG, Singapore (SG); Gaurav SAINI, Singapore (SG)

(21) Appl. No.: 19/404,069

(22) Filed: Dec. 1, 2025

Related U.S. Application Data
(60) Provisional application No. 63/726,807, filed on Dec. 2, 2024.
(60) Provisional application No. 63/796,486, filed on Apr. 29, 2025.

(51) Int. Cl. A61L 9/04; A61L 101/32

(52) U.S. Cl. CPC A61L 9/042; A61L 2101/32

(57) ABSTRACT

Freshening compositions include non-aldehyde perfume raw materials and a mixture of volatile aldehyde-containing compounds. The mixture of volatile aldehyde-containing compounds comprises at least a certain amount (e.g., 8% by weight of the freshening composition) of volatile aldehyde-containing compounds having a conjugated aldehyde group.

### FIELD OF THE INVENTION

The invention related to a volatile composition, especially to a volatile composition suitable for freshening an interior space, and to a volatile composition suitable for reducing malodor in an interior space. The invention also relates to devices and methods using the volatile composition.

### BACKGROUND OF THE INVENTION

Certain interior spaces in homes can contain malodor sources that result in a high concentration of malodor-causing molecules in the air. For example, food and other household waste in a kitchen or outdoor waste bin may result in a high concentration of malodor-causing molecules in the air. This results in unpleasant smells when a user opens the waste bin to deposit additional waste.

Although there exist products on the market for reducing malodor in a home, these come with drawbacks from the perspective of a waste bin and other enclosed interior spaces. Air fresheners in the form of sprays, candles, oils, and gels commonly use perfume to mask malodors, but are generally unable to reduce the presence of malodor-causing molecules in the air. Many anti-malodor products require active use, e.g., aerosol sprays that must be actively sprayed into the air or onto a surface. Due to their active nature, these products are unsuitable for counteracting waste bin malodor. Existing passive anti-malodor products such as plug-in electrical freshening products cannot be used inside a bin, and freestanding non-energized air freshening products are unsuitable for use inside a waste bin because they cannot adhere to the bin wall or lid, and so will become mixed up with the waste and disposed of when the waste is removed from the bin. Furthermore, such passive air freshening products on the market contain freshening compositions that are designed for different malodor sources, such as bathroom malodor.

U.S. Pat. No. 11,938,242 B2 relates to apparatus and methods involving the use of volatile carbonyl-containing compounds to reduce malodor on surfaces. However, this document does not discuss specific malodor sources, preferred types of volatile carbonyl-containing compounds, or tailoring a composition comprising volatile carbonyl-containing compounds to counteract any particular malodor source.

There is a need for a product that is able to effectively reduce waste malodor, especially food waste malodor, within a waste bin over multiple waste addition and removal cycles.

### SUMMARY OF THE INVENTION

The present inventors have surprisingly found that conjugated aldehyde-containing compounds having a vapor pressure of at least 0.018 torr at 25° C. have excellent efficacy at reducing malodor caused by food waste. This efficacy is substantially higher than the efficacy of non-conjugated aldehydes having equivalent vapor pressures.

A volatile composition comprising an increased amount of conjugated aldehyde-containing compounds having a vapor pressure of at least 0.018 torr at 25° C., results in improved reduction of malodor originating from food waste.

Therefore, the invention provides the following examples.
1. A freshening composition comprising:
   from about 0.01 wt. % to about 90 wt. % of one or more non-aldehyde perfume raw materials; and
   from about 10 wt. % to about 70 wt. % of a mixture of volatile aldehyde-containing compounds, the volatile aldehyde-containing compounds each having a vapor pressure of at least 0.018 Torr at 25° C.;
   wherein:
   the mixture of volatile aldehyde-containing compounds comprises at least 8%, by weight of the freshening composition, of volatile aldehyde-containing compounds having a conjugated aldehyde group.
2. The freshening composition according to claim 1, wherein the freshening composition comprises from about 12 wt. % to about 50 wt. % of the mixture of volatile aldehyde-containing compounds,
   optionally from about 15 wt. % to about 40 wt. %,
   more optionally from about 22 wt. % to about 40 wt. %.
3. The freshening composition according to claim 1 or 2, wherein the mixture of volatile aldehyde-containing compounds has a weight average vapor pressure of from about 0.33 Torr to about 0.6 Torr at 25° C.
4. The freshening composition according to any one of the previous claims, wherein the mixture of volatile aldehyde-containing compounds comprises at least 30%, by weight of the mixture of volatile aldehyde-containing compounds, of volatile aldehyde-containing compounds having a vapor pressure of greater than 0.1 Torr at 25° C.,
   optionally at least 35%,
   more optionally at least 40%.
5. The freshening composition according to any one of the previous claims, wherein the mixture of volatile aldehyde-containing compounds comprises at least 10%, by weight of the freshening composition, of volatile aldehyde-containing compounds having a conjugated aldehyde group.
6. The freshening composition according to any one of the previous claims, wherein:
   the freshening composition comprises at least 12%, by weight of the freshening composition, of the mixture of volatile aldehyde-containing compounds; and
   wherein the mixture of volatile aldehyde-containing compounds comprises at least 12%, by weight of the freshening composition, of volatile aldehyde-containing compounds having a conjugated aldehyde group.
7. The freshening composition according to any one of the previous claims, wherein a weight ratio of conjugated aldehyde-containing compounds to non-conjugated aldehyde-containing compounds in the mixture of volatile aldehyde-containing compounds is at least 1.05,
   optionally from 1.1 to 5,
   more optionally from 1.4 to 4.
8. The freshening composition according to any one of the previous claims, wherein the mixture of volatile aldehyde-containing compounds comprises at least 10 wt. %, based on the amount of the mixture of volatile aldehyde-containing compounds, of cinnamaldehyde, benzaldehyde, or a mixture thereof,
   optionally at least 15 wt. %,
   more optionally at least 17 wt. %.
9. The freshening composition according to any one of the previous claims, wherein the freshening composition has a weight average vapor pressure of from 0.37 to 0.6 Torr at 25° C., optionally wherein the freshening composition has a weight average vapor pressure of from 0.4 to 0.45 Torr at 25° C.
10. The freshening composition according to any one of the previous claims, wherein the one or more non-aldehyde perfume raw materials comprise at least 4 wt. %, by weight of the freshening composition, of non-aldehyde perfume raw materials having a citrus aroma.
11. A freshening composition comprising:
   from about 0.01 wt. % to about 90 wt. % of one or more non-aldehyde perfume raw materials; and
   from about 10 wt. % to about 70 wt. % of a mixture of volatile aldehyde-containing compounds, the volatile aldehyde-containing compounds each having a vapor pressure of at least 0.018 Torr at 25° C.;
   wherein:
   a weight ratio of conjugated aldehydes to non-conjugated aldehydes in the mixture of volatile aldehyde-containing compounds is at least 1.85:1.
12. A freshening composition comprising:
   from about 0.01 wt. % to about 90 wt. % of one or more non-aldehyde perfume raw materials; and
   from about 10 wt. % to about 70 wt. % of a mixture of volatile aldehyde-containing compounds, the volatile aldehyde-containing compounds each having a vapor pressure of at least 0.018 Torr at 25° C.;
   wherein:
   the mixture of volatile aldehyde-containing compounds comprises at least 5%, by weight of the freshening composition, of volatile aldehyde-containing compounds having a conjugated aldehyde group; and
   the mixture of volatile aldehyde-containing compounds comprises at least 5%, by weight of the freshening composition, of volatile aldehyde-containing compounds having a vapor pressure of at least 0.1 Torr at 25° C.
13. An air freshening product comprising:
   a reservoir enclosed by a membrane, the reservoir containing the freshening composition according to any one of the preceding claims, where the freshening composition is configured to evaporate through or from the membrane,
   wherein the air freshening product may optionally require an activation step to enable evaporation of the freshening composition through or from the membrane.
14. A method of reducing malodor in a waste bin, comprising:
   placing an air freshening product according to claim 13 into or adjacent to the waste bin;
   optionally activating the air freshening product; and
   allowing the freshening composition to evaporate from the air freshening product.
15. Use of a freshening composition according to any one of claims 1 to 12, or of an air freshening product according to claim 13, to reduce malodor in a waste bin.

### DETAILED DESCRIPTION OF THE INVENTION

The invention, in an embodiment, relates to a freshening composition comprising:
from about 0.01 wt. % to about 90 wt. % of one or more non-aldehyde perfume raw materials; and
from about 10 wt. % to about 70 wt. % of a mixture of volatile aldehyde-containing compounds, the volatile aldehyde-containing compounds each having a vapor pressure of at least 0.018 Torr at 25° C.;
wherein:
the mixture of volatile aldehyde-containing compounds comprises at least 8%, by weight of the freshening composition, of volatile aldehyde-containing compounds having a conjugated aldehyde group.

The invention, in an embodiment, also relates to a freshening composition comprising:
from about 0.01 wt. % to about 90 wt. % of one or more non-aldehyde perfume raw materials; and
from about 10 wt. % to about 70 wt. % of a mixture of volatile aldehyde-containing compounds, the volatile aldehyde-containing compounds each having a vapor pressure of at least 0.018 Torr at 25° C.;
wherein:
a weight ratio of conjugated aldehydes to non-conjugated aldehydes in the mixture of volatile aldehyde-containing compounds is at least 1.85:1.

The invention, in an embodiment, also relates to a freshening composition comprising:
from about 0.01 wt. % to about 90 wt. % of one or more non-aldehyde perfume raw materials; and
from about 10 wt. % to about 70 wt. % of a mixture of volatile aldehyde-containing compounds, the volatile aldehyde-containing compounds each having a vapor pressure of at least 0.018 Torr at 25° C.;
wherein:
the mixture of volatile aldehyde-containing compounds comprises at least 5%, by weight of the freshening composition, of volatile aldehyde-containing compounds having a conjugated aldehyde group; and
the mixture of volatile aldehyde-containing compounds comprises at least 5%, by weight of the freshening composition, of volatile aldehyde-containing compounds having a vapor pressure of at least 0.1 Torr at 25° C.

As used herein, the term “freshening composition”, or equivalent, refers to a composition that can function as an air freshener, an odor reducing composition, an odor neutralizing composition, a malodor counteractant, or a combination thereof.

The freshening compositions defined above may collectively be referred to as “the freshening composition”, “the freshening composition of the invention”, or plural versions thereof. For the sake of brevity, these phrases may be shortened to simply “the composition”, or “the composition of the invention”, or plural versions thereof. The use of any of these phrases, or equivalent, is intended to refer to each and all of the freshening compositions defined above.

As used herein, references to “reducing”, “counteracting”, or “neutralising” odor or malodor refer to the ability of a volatile aldehyde-containing compound to reduce or eliminate the presence of malodor-causing compounds (e.g., amines and thiols). Reduction of malodor may be partial, affecting only some of the malodor-causing compounds in a given context; or it may be complete, affecting all of the malodor-causing compounds in said context. Typically, a malodor-causing compound such as an amine or a thiol may be neutralized by chemical reaction with an aldehyde resulting in a new chemical entity, which new chemical entity does not cause malodor. This effect may be distinguished from odor masking or odor blocking because it results in a chemical change of the malodor-causing compound, as opposed to a change in the ability of a person to perceive the malodor without any corresponding change of the malodor-causing compound. Nevertheless, the freshening composition of the invention may additionally comprise odor masking or odor blocking materials, to further reduce the perception of malodor.

The freshening composition of the invention is able to volatilise at room temperature and atmospheric pressure without the need of an energy source. Typically, the freshening composition is in the form of a liquid at 25° C. and atmospheric pressure. Advantageously, this allows the freshening composition to become impregnated into a microporous membrane, and to evaporate/volatilise therefrom. Nevertheless, the freshening composition of the invention may be allowed to volatilise by any appropriate means, such as from a wick, reed diffuser, polymer gel, through a monolithic membrane, and through or from a microporous membrane.

It should also be understood that when the freshening composition is described herein as being “volatilised”, “evaporated”, “delivered”, “emitted”, or “released”, this refers to the volatilization of the volatile component thereof (e.g., the mixture of volatile aldehyde-containing compounds, and the one or more non-aldehyde perfume raw materials), and does not require that any non-volatile components thereof be emitted. As used herein, the term “vaporize” or “vaporization” refers to a phase transition of a substance or a compound from a solid and/or liquid phase to vapor.

As used herein, a “perfume raw material” or “PRM” refers to one or more of the following ingredients: fragrant essential oils; aroma compounds; pro-perfumes; materials supplied with the fragrant essential oils, aroma compounds, including stabilizers, diluents, processing agents, and contaminants; and any material that commonly accompanies fragrant essential oils and aroma compounds. Particular examples of fragrant essential oils include but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like.

The freshening composition of the invention may include multiple perfume raw materials, which may have similar, related, complementary, or contrasting smells.

A “non-aldehyde perfume raw material” generally refers to a perfume raw material as defined above that does not contain an aldehyde functional group. A non-aldehyde perfume raw material does not include volatile aldehyde-containing compounds having a vapor pressure of at least 0.018 Torr at 25° C.

Particular examples of non-aldehyde perfume raw materials include but are not limited to: cyclic ethylene dodecanedioate, 4-tertiary butyl cyclohexyl acetate or Vertenex™, allyl amyl glycolate, allyl caproate, allyl cyclohexane propionate, allyl heptanoate, amber xtreme, ambrox, isoamyl acetate, isoamyl propionate, anethole usp, benzyl acetate, benzyl propionate, cis-3-hexen-1-ol, beta naphthol methyl ether or nerolin, caramel furanone, caryophyllene extra, Cinnamalva™ or Cinnamyl Nitrile, cinnamyl acetate, cinnamyl nitrile, cis-3-hexenyl butyrate, cis-3-hexenyl acetate, cis-3-hexenyl alpha methyl butyrate, cis-6-nonen-1-ol, citrathal or citral diethyl acetal, citronellol, citronellyl acetate, citronellyl butyrate, clonal or dodecane nitrile, coranol or 2,2-dimethyl cyclohexanepropanol, coumarin, cumin nitrile, cuminic alcohol, tricyclodecenyl isobutirate or cyclabute, cyclohexyl ethyl acetate, dihydromyrcenol, dimethyl anthranilate, dimethyl benzyl carbinyl acetate, dimethyl-2 6-heptan-2-ol or freesiol, sandal pentenol or ebanol, ethyl-2-methyl pentanoate, ethyl acetoacetate, ethyl linalool, ethyl maltol, ethyl phenyl glycidate, ethyl vanillin, ethyl-2-methyl butyrate, eucalyptol, eugenol, flor acetate, ozone propanal or floralozone, Fructalate™ or raspberry dicarboxylate, geraniol or trans-3,7-dimethyl-2,7-octadien-1-ol, Grisalva™ or amber furan, Habanolide™ or (E)-12-musk decenone, Helvetolide™ or musk propanoate, hexyl acetate, hexyl-2-methyl butyrate, Indocolore™ or 1-phenylvinyl acetate, iso bornyl acetate, iso eugenyl acetate, iso propyl myristate, isoamyl butyrate, isoeugenol, Koumalactone™ or dihydromint lactone, laevo trisandol or sandranol, Lemonile™ or homogeranyl nitrile, Levistamel™ or mesitene lactone, linalool, linalyl acetate, linalyl iso butyrate, lymolene or dihydromyrcenol, menthol, methyl dioxolan or Fructone™, methyl iso butenyl tetrahydro pyran, methyl Pamplemousse™ or grapefruit acetal, methyl phenyl carbinyl acetate or styrallyl acetate, methyl salicylate, Montaverdi™ or green cyclopropionate, Mugetanol™ or muguet ethanol, neocaspirene, neofolione or melon nonenoate nerolidol, orange terpenes, orcinyl-3 or 3-methoxy-5-methylphenol, Oxane™ or cis-galbanum oxathiane, para cresyl methyl ether or para methyl anisole, patchouli, phenyl ethyl alcohol, phenyl ethyl dimethyl carbinol, Polysantol™ or santol pentenol, prenyl acetate, Sauvignone™ or 5-mercapto-5-methyl-3-hexanone, Sclareolate™ or clary propionate, shisolia, Strawberiff™ or 2-methyl-2-pentenoic acid, terpinolene or 4-isopropylidene-1-methylcyclohexene, tetrahydro Muguol™ or citrus ocimenol, Thesaron™ (1R,6S)-2,2,6-Trimethyl-cyclohexanecarboxylic acid ethyl ester, Tobacarol™ or 5-tetramethyl oxatricyclododecane, Undecavertol™ or violet decenol, Verdox™ or green acetate, Verdural B™ or (Z)-3-hexen-1-yl isobutyrate, Violettyne™ or violet dienyne, Violiff™ or violet methyl carbonate, and mixtures thereof.

The freshening composition comprises a mixture of volatile aldehyde-containing compounds, and one or more non-aldehyde perfume raw materials. The freshening composition may also include additional components, such as solvents and preservatives, which additional components may be volatile or non-volatile.

As will be appreciated by a person skilled in the art, one or more perfume raw materials included in the freshening composition may also have solvent-like properties, and assist in the dissolution of components of the freshening composition that might otherwise be poorly soluble.

One or more solvents (e.g., one or more substantially non-odorous solvents) may be included to improve the properties of the freshening composition. For example, solvents may be included to modify the viscosity, surface tension, or evaporative properties of the freshening composition. The freshening composition may include any appropriate solvent, in any appropriate amount to provide the desired properties. The freshening composition may contain a single solvent or a mixture of solvents. Suitable solvents include, but are not limited to: 1,2-isopropylideneglycerol, dipropylene glycol, 3-methoxy-3-methylbutan-1-ol (MMB), 3-methylbutan-2-ol, butan-1-ol, 2,3-dimethylbutan-2-ol, 1-methoxypropan-2-ol, 2-methylbutan-2-ol, 3-methylbutan-1-ol, hex-1-en-3-ol, 2-ethylbutan-1-ol, 4-methylpentan-1-ol, 3-methylpentan-1-ol, ethyl 2-hydroxypropanoate, 2-butoxyethanol, ethyl 3-hydroxybutanoate, dimethyl hexanedioate (DMA), ethyl 3,5,5-trimethylhexanoate, dimethyl butanedioate, diethyl propanedioate, ethyl 3-acetyloxyhexanoate, methyl 5-acetyloxyhexanoate, 3-O-butyl 1-O-ethyl propanedioate, dipropan-2-yl hexanedioate, (4-methoxyphenyl)methyl formate, ethyl 3-hydroxyhexanoate, 1-(3-methoxypropoxy)propan-1-ol (DPM), 2-(2-Methoxyethoxy)ethanol, methyl 2-hydroxybenzoate, 6,8-dimethylnonan-2-ol, 2-phenoxyethanol, 4-Oxa-1,6-hexandiol,1-(1-methyl-2-propoxyethoxy)propan-2-ol, 1-(2-butoxy-1-methoxy)propan-2-ol, and mixtures thereof.

In some configurations, a mixture of solvents having different vapor pressures may be used to modulate the evaporation of the volatile composition, as described in WO 2022/006198 A1 and WO 2022/006197 A1.

In some configurations, the freshening composition may have a viscosity of from about 1.0 cP to about 25 cP, such as from about 1.0 cP to about 23 cP, such as from about 1.0 cP to about 15 cP. In some configurations, the freshening composition may have a surface tension of from about 19 mN/m to about 33 mN/m, such as from about 19 mN/m to about 30 mN/m, such as from about 19 mN/m to about 27 mN/m.

In some configurations, the freshening composition may be substantially free of water, e.g., free of water. In some configurations, the freshening composition may comprise less than 5% water, by weight of the freshening composition. In some configurations, the freshening composition may comprise less than 4% water, by weight of the freshening composition. In some configurations, the freshening composition may comprise less than 3% water, by weight of the freshening composition. In some configurations, the freshening composition may comprise less than 2% water, by weight of the freshening composition. In some configurations, the freshening composition may comprise less than 1% water, by weight of the freshening composition. In some configurations, the freshening composition may comprise less than 0.5% water, by weight of the freshening composition. In some configurations, the freshening composition may comprise less than 0.1% water, by weight of the freshening composition.

The freshening composition of the invention comprises a mixture of volatile aldehyde-containing compounds. The mixture of volatile aldehyde-containing compounds may consist of a single volatile aldehyde-containing compound, or more typically, may comprise multiple volatile aldehyde-containing compounds. The volatile aldehyde-containing compounds of the mixture of volatile aldehyde-containing compounds each have a vapor pressure of at least 0.018 Torr at 25° C. For the purposes of the present invention, aldehyde-containing compounds having a vapor pressure of less than 0.018 Torr at 25° C. are not considered to be volatile aldehyde-containing compounds.

For the avoidance of doubt, the freshening composition of the invention may nevertheless include aldehyde-containing compounds that have a vapor pressure of less than 0.018 Torr at 25° C. Such compounds are not considered part of the mixture of volatile aldehyde-containing compounds, but may be present, for example, as perfume raw materials.

The composition may be used in air freshening products or devices that are configured to release the composition into a surrounding environment. While the composition is particularly adapted for use inside a waste bin, and so may typically be used within a continuous non-energized air freshening device, the composition may nevertheless be used with active (e.g., aerosol) or energised devices that require an electrical power source.

When the composition of the invention is volatilised, the aldehyde-containing compounds of the mixture of volatile aldehyde-containing compounds are volatilised and able to react with malodor-causing molecules in the air, and also deposit on surfaces to react with malodor-causing molecules adsorbed onto the surface, as described in WO 2019/089739 A1.

The composition of the invention comprises a higher amount of volatile conjugated aldehyde-containing compounds, as compared to prior art compositions. Without being bound by theory, it is believed that food waste contains an increased level of sulfurous malodor-causing molecules (e.g., thiols), as compared to other malodor sources, such as bathroom malodor which is predominantly caused by amines. While food waste contains both thiols and amines, thiols are generally more potent malodor-causing compounds and are detected by the human olfactory system at lower concentrations than amines. It is believed that the composition of the invention, containing an increased amount of conjugated aldehyde-containing compounds, is able to better neutralise malodor caused by thiols. Thiols are soft nucleophiles and so may react better with a conjugated aldehyde, because an aldehyde having a conjugated alkene group may act as a soft electrophile (by 1,4-nucleophilic addition) and/or because conjugated aldehydes have a lower energy LUMO that better matches the HOMO of a thiol (e.g., in the case of aldehydes conjugated to phenyl rings). In contrast, the carbonyl carbon of a non-conjugated aldehyde may preferentially react with harder nucleophiles, such as amines, and so be less effective a reducing malodor caused by thiols.

Tables 1 and 2 below list examples of suitable conjugated and non-conjugated aldehydes that may be useful in the composition of the invention.

TABLE 1
Conjugated aldehydes

| CAS | IUPAC Name |
|---|---|
| 6728-26-3 | (E)-hex-2-enal |
| 2363-89-5 | (E)-oct-2-enal |
| 2463-53-8 | non-2-enal |
| 557-48-2 | (2E,6Z)-nona-2,6-dienal |
| 100-52-7 | Benzaldehyde |
| 04-55-2 | (E)-3-phenylprop-2-enal (Cinnamaldehyde) |
| 5392-40-5 | 3,7-dimethylocta-2,6-dienal (Citral) |
| 122-03-2 | 4-propan-2-ylbenzaldehyde (Cuminic aldehyde) |
| 123-11-5 | 4-methoxybenzaldehyde (Anisic aldehyde) |

Of the conjugated aldehydes listed in Table 1, Citral, Benzaldehyde and Cinnamaldehyde are particularly effective at reducing malodor in a waste bin and providing consumer satisfaction.

It is believed that benzaldehyde and cinnamaldehyde, having low energy LUMOs, are able to react effectively with malodor-causing thiol compounds released by food waste. These compounds are not suitable for use in generic air freshening products, because cinnamaldehyde has a spicy aroma and benzaldehyde has a strong almond scent. These aromas make it impractical to incorporate benzaldehyde and cinnamaldehyde into freshening products intended for use inside interior spaces such as rooms, cars, or for freshening fabrics. Nevertheless, these downsides do not apply to uses in a waste bin, where relatively higher amounts of cinnamaldehyde and benzaldehyde may be used to effectively reduce malodor caused by food waste. Therefore, in some configurations the mixture of volatile aldehyde-containing compounds comprises benzaldehyde and/or cinnamaldehyde. In some configurations, the mixture of volatile aldehyde-containing compounds comprises at least 10 wt. % (optionally at least 15 wt. %, such as at least 17 wt. %), based on the amount of the mixture of volatile aldehyde-containing compounds, of cinnamaldehyde, benzaldehyde, or a mixture thereof.

Citral, being a conjugated aldehyde, is also able to effectively react with thiol compounds, but in addition has a citrus aroma, which consumers typically associate with clean and sanitary conditions. The inclusion of Citral in the composition of the invention may help provide a citrus aroma to the freshening composition, leading to increased consumer satisfaction. Thus, in some configurations the mixture of volatile aldehyde-containing compounds comprises citral. In some configurations, the mixture of volatile aldehyde-containing compounds comprises at least 10 wt. % (optionally at least 20 wt. %), based on the amount of the mixture of volatile aldehyde-containing compounds, of citral.

In some configurations, the mixture of volatile aldehyde-containing compounds comprises at least 10 wt. % (optionally at least 17 wt. %), based on the amount of the mixture of volatile aldehyde-containing compounds, of cinnamaldehyde, benzaldehyde, or a mixture thereof, and comprises at least 10 wt. % (optionally at least 20 wt. %), based on the amount of the mixture of volatile aldehyde-containing compounds, of citral.

TABLE 2
Non-conjugated aldehydes

| CAS | IUPAC Name |
|---|---|
| 1335-66-6 | 2,4,6-trimethylcyclohex-3-ene-1-carbaldehyde; 3,5,6- |
| | trimethylcyclohex-3-ene-1-carbaldehyde |
| 124-13-0 | Octanal |
| 68039-49-6 | 2,4-dimethylcyclohex-3-ene-1-carbaldehyde |
| 106-72-9 | 2,6-dimethylhept-5-enal |
| 124-19-6 | Nonanal |
| 65405-70-1 | (E)-dec-4-enal |
| 2277-19-2 | (Z)-non-6-enal |
| 106-23-0 | 3,7-dimethyloct-6-enal |
| 62439-41-2 | 6-methoxy-2,6-dimethylheptanal |
| 112-31-2 | Decanal |
| 6784-13-0 | 3-(4-methylcyclohex-3-en-1-yl)butanal |
| 19009-56-4 | 2-methyldecanal |
| 3613-30-7 | 7-methoxy-3,7-dimethyloctanal |
| 33885-51-7 | 3-(6,6-dimethyl-4-bicyclo[3.1.1]hept-3-enyl)propanal |
| 112-44-7 | Undecanal |
| 33885-52-8 | 3-(6,6-dimethyl-4-bicyclo[3.1.1]hept-3-enyl)-2,2- |
| | dimethylpropanal |
| 112-45-8 | undec-10-enal |
| 71077-31-1 | 4,8-dimethyldeca-4,9-dienal |
| 143-14-6 | undec-9-enal |

In some configurations, the freshening composition may comprise from about 12 wt. % to about 50 wt. % of the mixture of volatile aldehyde-containing compounds. In some configurations, the freshening composition may comprise from about 15 wt. % to about 40 wt. % of the mixture of volatile aldehyde-containing compounds. In some configurations, the freshening composition may comprise from about 22 wt. % to about 40 wt. % of the mixture of volatile aldehyde-containing compounds.

As discussed herein, the composition of the invention may be particularly suitable for freshening the interior of waste bins, especially food/kitchen waste bins. In order to ensure that the mixture of volatile aldehyde-containing compounds has appropriate volatility to counteract malodor in a relatively low airflow environment of a waste bin, the mixture of volatile aldehyde-containing compounds may have a weight average vapor pressure of from about 0.33 Torr to about 0.6 Torr at 25° C. Alternatively, or in addition, the mixture of volatile aldehyde-containing compounds may comprise at least 30%, by weight of the mixture of volatile aldehyde-containing compounds, of volatile aldehyde-containing compounds having a vapor pressure of greater than 0.1 Torr at 25° C., optionally at least 35%, more optionally at least 40%.

In order to improve the ability of the composition of the invention to counteract malodor, the mixture of volatile aldehyde-containing compounds may comprise at least 10%, by weight of the freshening composition, of volatile aldehyde-containing compounds having a conjugated aldehyde group. In some configurations, the mixture of volatile aldehyde-containing compounds may comprise at least 12%, by weight of the freshening composition, of volatile aldehyde-containing compounds having a conjugated aldehyde group.

In order to improve the ability of the composition of the invention to counteract malodor, without using an excessive amount of volatile aldehyde-containing compounds, the composition may include a greater amount of volatile conjugated aldehyde-containing compounds than volatile non-conjugated aldehyde-containing compounds. Therefore, in some configurations, a weight ratio of conjugated aldehyde-containing compounds to non-conjugated aldehyde-containing compounds in the mixture of volatile aldehyde-containing compounds may be least 1.05, e.g., at least 1.1, or at least 1.4. In some configurations, a weight ratio of conjugated aldehydes to non-conjugated aldehydes in the mixture of volatile aldehyde-containing compounds may be from 1.1 to 5, such as from 1.4 to 4.

In order to ensure that the aroma of the composition of the invention is noticeable to a consumer when opening a waste-bin lid, the composition may have a weight average vapor pressure that ensures sufficient volatilisation within low airflow environments. Therefore, in some configurations, the freshening composition may have a weight average vapor pressure of from 0.37 to 0.6 Torr at 25° C., such as from 0.4 to 0.5 Torr at 25° C., or from 0.4 to 0.45 Torr at 25° C.

As mentioned above, consumers may associate a citrus aroma with a clean and sanitary environment. Therefore, it may be advantageous and increase consumer satisfaction for the composition of the invention to have a citrus aroma. Thus, in some configurations the one or more non-aldehyde perfume raw materials comprise at least 4% (e.g., at least 6%, or at least 10%), by weight of the freshening composition, of non-aldehyde perfume raw materials having a citrus aroma. Specific examples of non-aldehyde perfume raw materials having a citrus aroma include 2-tridecenenitrile, dihydro myrcenol, 2,6-dimethylheptan-2-ol, 4-(2-methoxypropan-2-yl)-1-methylcyclohexene, 3,7-dimethylnona-2,6-dienenitrile, 2-(4-methyl-1-cyclohex-3-enyl)propane-2-thiol, orange oil terpenes (CAS 68647-72-3), 3,7-dimethyloct-6-enenitrile, 1-methyl-4-propan-2-ylcyclohexa-1,4-diene, citrus aurantium (CAS 68514-75-0), 6,6-dimethoxy-2,5,5-trimethylhex-2-ene, 1-methyl-4-propan-2-ylidenecyclohexene, and mixtures thereof. Other (non-aldehyde) perfume raw materials having a citrus aroma include those classified under “Primary” or “Secondary” citrus odor descriptors by The Good Scents Company Information System, available from The Good Scents Company at http://www.thegoodscentscompany.com/odor/citrus.html.

In some configurations, the mixture of volatile aldehyde-containing compounds may comprise at least 20% (e.g., at least 30%, or at least 40%), by weight of the mixture of volatile aldehyde-containing compounds, of aldehyde-containing compounds having a citrus aroma. Examples of aldehyde-containing compounds having a citrus aroma are listed below in Table 3.

TABLE 3
Volatile aldehyde-containing compounds having a citrus aroma

| | | Conjugated or |
|---|---|---|
| CAS | IUPAC Name | non-conjugated |
| 124-13-0 | Octanal | Non-conjugated |
| 124-19-6 | Nonanal | Non-conjugated |
| 65405-70-1 | (E)-4-decenal | Non-conjugated |
| 2385-77-5 | (3R)-3,7-dimethyloct-6-enal | Non-conjugated |
| | (Citronellal) | |
| 112-31-2 | Decanal | Non-conjugated |
| 112-44-7 | Undecanal | Non-conjugated |
| 5392-40-5 | 3,7-dimethyl-2,6-octadienal (Citral) | Conjugated |
| 55722-59-3 | 3,7-dimethylocta-3,6-dienal (Isocitral) | Non-conjugated |
| 899810-84-5 | 6,8-dimethylnon-7-enal (Noreenal) | Non-conjugated |
| 6784-13-0 | 3-(4-methyl-1-cyclohex-3-enyl)butanal | Non-conjugated |
| | (Liminal ®) | |

Other aldehyde-containing compounds having a citrus aroma include volatile aldehydes classified under “Primary” or “Secondary” citrus odor descriptors by The Good Scents Company Information System, available from The Good Scents Company at http://www.thegoodscentscompany.com/odor/citrus.html.

In addition to the mixture of volatile aldehyde-containing compounds, the freshening composition may comprise one or more volatile ketone-containing compounds. While ketone-containing compounds are typically less reactive than aldehyde-containing compounds, they may also react with amine- and thiol-containing malodor-causing compounds.

Examples of ketone-containing compounds include but are not limited to: 2,6,6-trimethylcyclohex-2-ene-1,4-dione, 4,7,7-trimethylbicyclo[2.2.1]heptane-2,3-dione, 1-(5-methylfuran-2-yl)ethanone 3-methylcyclopentane-1,2-dione, 1-phenylethanone, pentane-2,3-dione, 4-methoxy-2,5-dimethylfuran-3-one, 4-hydroxy-2,5-dimethylfuran-3-one, (1S,5S)-2,6,6-trimethylbicyclo[3.1.1]hept-2-en-4-one, (1R,5R)-2,6,6-trimethylbicyclo[3.1.1]hept-2-en-4-one, 3,5,5-trimethylcyclohex-2-en-1-one, 3-methylcyclopent-2-en-1-one, (5S)-2-methyl-5-prop-1-en-2-ylcyclohex-2-en-1-one, (5R)-2-methyl-5-prop-1-en-2-ylcyclohex-2-en-1-one, 4-methylpent-3-en-2-one, 2-methyl-5-prop-1-en-2-ylcyclohex-2-en-1-one, 1-(1H-pyrrol-2-yl)ethenone, (5R)-5-methyl-2-propan-2-ylidenecyclohexan-1-one, 4-phenylbutan-2-one, 3-methylbutyl 3-oxobutanoate, 3-hydroxybutan-2-one, (Z)-3,4,5,6,6-pentamethylhept-3-en-2-one, 3-oxobutan-2-yl acetate, methyl 3-oxobutanoate, ethyl 3-oxobutanoate, (2R,5R)-2-methyl-5-prop-1-en-2-ylcyclohexan-1-one, 2-methyl-5-prop-1-en-2-ylcyclohexan-1-one, (2S,5S)-2-methyl-5-prop-1-en-2-ylcyclohexan-1-one, 2-ethyl-4,4-dimethylcyclohexan-1-one, 1-(3,3-dimethylcyclohexyl)ethenone, (2S,5S)-5-methyl-2-propan-2-ylcyclohexan-1-one, (2S,5R)-5-methyl-2-propan-2-ylcyclohexan-1-one, 2,2,5-trimethyl-5-pentylcyclopentan-1-one, 3,3,5-trimethylcyclohexan-1-one, 2-cyclopentylcyclopentan-1-one, (1S,4R,5R)-4-methyl-1-propan-2-ylbicyclo[3.1.0]hexan-3-one, 4-(2-methylbutan-2-yl)cyclohexan-1-one, 4,7,7-trimethylbicyclo[2.2.1]heptan-3-one, 6-methylhept-5-en-2-one, octan-2-one, (1S,4R)-2,2,4-trimethylbicyclo[2.2.1]heptan-3-one, heptan-2-one, 2,2,4-trimethylbicyclo[2.2.1]heptan-3-one, 5-methylheptan-3-one, octan-3-one, and mixtures thereof.

The freshening composition may comprise one or more additional components selected from an anti-bacterial component, an anti-mold component, and an anti-insect component (e.g. an insect repellant or an insecticide), any of which may be present in any effective amount.

### Anti-Bacterial Component

The anti-bacterial component may be any suitable compound or mixture. In some configurations, the anti-bacterial component may comprise a mixture of volatile aldehydes that consists of
(i) a C5 to C8 unbranched unsubstituted linear alkenal; and
(ii) a C9 to C14 unbranched unsubstituted linear alkenal,

wherein a weight ratio of the C5 to C8 unbranched unsubstituted linear alkenal to the C9 to C14 unbranched unsubstituted linear alkenal is from 3:1 to 1:3 (e.g. 2:1 to 1:2). Components (i) and (ii) may be present in a combined amount of at least 0.5 wt. %, based on the weight of the freshening composition. The alkenals may have double bonds having a trans configuration. Specific examples of C5 to C8 unbranched unsubstituted linear alkenals include 2-penten-1-al, 3-penten-1-al, 4-penten-1-al, (E)-2-hexen-1-al, 3-hexen-1-al, 4-hexen-1-al, 2-hepten-1-al, 3-hepten-1-al, 4-hepten-1-al, 2-octen-1-al, 3-octen-1-al, 4-octen-1-al, and mixtures thereof. Specific examples of C9 to C14 unbranched unsubstituted linear alkenals include 2-nonen-1-al, 3-nonen-1-al, 4-nonen-1-al, 5-nonen-1-al, 2-decen-1-al, 3-decen-1-al, 4-decen-1-al, 5-decen-1-al, 2-undecen-1-al, 3-undecen-1-al, 4-undecen-1-al, 5-undecen-1-al, 2-dodecen-1-al, 3-dodecen-1-al, 4-dodecen-1-al, 5-dodecen-al-1, 2-tridecen-1-al, 3-tridecen-1-al, 4-tridecen-1-al, 2-tetradecen-1-al, 3-tetradecen-1-al, 4-tetradecen-1-al, 5-tetradecen-1-al and mixtures thereof.

Anti-mold Component The anti-mold component may be any suitable compound or mixture. In some configurations, the anti-bacterial component may comprise:
(i) a C5 to C8 unbranched unsubstituted linear alkenal; and
(ii) a C9 to C14 unbranched unsubstituted linear alkenal,

wherein the C9-C14 unbranched unsubstituted linear alkenal comprises a single double bond at the C3, C4, or C5 position. Components (i) and (ii) may be present in a combined amount of at least 0.4 wt. %, with at least 0.2 wt. % of each of component (i) and (ii), based on the weight of the freshening composition.

The freshening compositions disclosed herein may be released from any appropriate device or apparatus. Such a device or apparatus may comprise a solid delivery member configured to contain the freshening composition in a liquid phase, and allow the freshening composition to evaporate or volatilise therefrom. For example, the delivery member may comprise a wick, a breathable membrane, a gel, a porous or semi-porous substrate, such as a felt or fabric pad. An exemplary delivery member may be a membrane that allows the freshening composition to pass through in a vapor phase, but prevents the passage of liquid freshening composition. Such membranes may be microporous or monolithic. The freshening composition may be impregnated into the membrane, from which it may evaporate, without leaking through the membrane in liquid form. Suitable membranes are described in, for example, US patent application publication no. US 2025/058008A1 and U.S. Pat. No. 11,938,242 B2.

In a further aspect, the invention provides an air freshening product comprising:
a reservoir enclosed by a membrane, the reservoir containing the freshening composition as defined herein, where the freshening composition is configured to evaporate through or from the membrane,
wherein the air freshening product may optionally require an activation step to enable evaporation of the freshening composition through or from the membrane.

The air freshening product may have any appropriate form, and may comprise a reusable housing into which a cartridge comprising the freshening composition may be placed. The cartridge may comprise a membrane enclosing a reservoir containing the freshening composition, such that the freshening composition may evaporate through or from the membrane. Such cartridges and devices are described in, for example, PCT patent application publication no. WO 2024/253990 A1, US patent application publication no. US 2024/398094 A1, and U.S. provisional patent application No. 63/677,126 (published as part of the file wrapper of priority-claiming applications), and its corresponding non-provisional patent application no. U.S. Ser. No. 19/282,107.

In a further aspect, the invention provides method of reducing malodor in a waste bin, comprising:
placing an air freshening product as defined herein into or adjacent to the waste bin;
optionally activating the air freshening product; and
allowing the freshening composition to evaporate from the air freshening product.

In a further aspect, the invention provides the use of a freshening composition as defined herein to reduce malodor in a waste bin (e.g., to reduce the concentration of one or more malodor-causing thiols or amines in a waste bin).

In a further aspect, the invention provides the use of an air freshening product as defined herein to reduce malodor in a waste bin (e.g., to reduce the concentration of one or more malodor-causing thiols or amines in a waste bin).

Reduction of malodor in a waste bin may be determined by any appropriate method, such as by Sensory Malodor Test Method 1 or Sensory Malodor Test Method 2, both of which are defined in the below Examples. The reduction in the concentration of one or more malodor-causing thiols or amines in a waste bin may be determined by any appropriate method, such as Analytical Malodor Test Method 1 or Analytical Malodor Test Method 2, both of which are defined in the below Examples. The reduction may be measured over any appropriate time period, such as 1 hour, 3 hours, 24 hours, 1 week, 2 weeks, 4 weeks, or 6 weeks, and may be determined by comparison against a control sample that does not include the freshening composition defined herein.

Also provided herein is a method of reducing malodor in a waste bin, the method comprising:
(a) receiving a cartridge comprising a reservoir containing a freshening composition, a membrane enclosing the reservoir, and a removable sealing substrate enclosing the membrane;
(b) removing the removable sealing substrate from the cartridge and placing the removable sealing substrate into a wase bin, where the removable sealing substrate contains residue of the freshening composition,

wherein the freshening composition comprises at least 10 wt. % (e.g. at least 12 wt. %, such as at least 15 wt. %, at least 20 wt. % or at least 25 wt. %), based on the weight of the freshening composition, of a mixture of volatile aldehyde-containing compounds, the volatile aldehyde-containing compounds each having a vapor pressure of at least 0.018 Torr at 25° C. In some configurations, the mixture of volatile aldehyde-containing compounds may comprise at least 8 wt. %, by weight of the freshening composition, of volatile aldehyde-containing compounds having a conjugated aldehyde group. The mixture of volatile aldehyde-containing compounds may have any feature or combination of features described hereinabove.

Such a method advantageously provides immediate anti-odor benefits, because the residual freshening composition on the sealing substrate will rapidly evaporate from the sealing substrate in the waste bin, due to a very high surface area:volume ratio. This provides an immediate reduction in malodor, increasing consumer satisfaction. In some configurations, the sealing substrate, when removed from the volatile composition cartridge, may comprise from 2 to 10 mg of freshening composition.

Also provided herein is a kit comprising: (i) a volatile composition cartridge comprising a reservoir containing a freshening composition, a membrane enclosing the reservoir, and a removable sealing substrate enclosing the membrane; and (ii) instructions for disposing of the sealing substrate in a waste bin, wherein the freshening composition comprises at least 10 wt. % (e.g. at least 12 wt. %, such as at least 15 wt. %, at least 20 wt. % or at least 25 wt. %), based on the weight of the freshening composition, of a mixture of volatile aldehyde-containing compounds, the volatile aldehyde-containing compounds each having a vapor pressure of at least 0.018 Torr at 25° C. In some configurations, the mixture of volatile aldehyde-containing compounds may comprise at least 8 wt. %, by weight of the freshening composition, of volatile aldehyde-containing compounds having a conjugated aldehyde group. The mixture of volatile aldehyde-containing compounds may have any feature or combination of features described hereinabove. By ensuring the sealing substrate with residual freshening composition is disposed of in accordance with the instructions, an improved anti-odor effect can be obtained. While there already exist cheap air-freshening products having this general construction, these cheap products do not include volatile aldehydes to provide an anti-malodor effect. The disclosed kit can therefore provide improved anti-malodor effect by combining the mixture of volatile aldehyde-containing compounds disclosed herein with the disposable sealing substrate, to provide both an immediate anti-malodor effect from evaporation of the residual freshening composition, and a long-lasting anti-malodor effect from release of the freshening composition through the membrane over time.

Also provided herein is a method of reducing or preventing malodor in a waste bin, the method comprising:
(i) receiving an air freshening product comprising a freshening composition that comprises at least 10 wt. %, based on the weight of the freshening composition, of a mixture of volatile aldehyde-containing compounds, the volatile aldehyde-containing compounds each having a vapor pressure of at least 0.018 Torr at 25° C.; and
(ii) placing the air freshening product on an interior surface of the waste bin and allowing the freshening composition to volatilise from the air freshening product to provide a concentration of volatile aldehyde containing compounds within a headspace of the waste bin of at least 2 ppm, such as at least 2.5 ppm, or at least 3 ppm (e.g. from about 2 ppm to about 10 ppm, such as about 2.5 ppm to about 7 ppm, or about 3 ppm to about 6 ppm).

In some configurations, at least half of the concentration of volatile aldehyde containing compounds within a headspace of the waste bin corresponds to the concentration of conjugated aldehyde containing compounds. For example, in the above method, the freshening composition may comprise volatile conjugated aldehyde containing compounds, and step (ii) may provide a concentration of volatile conjugated aldehyde containing compounds within a headspace of the waste bin of at least 1 ppm, such as at least 1.25 ppm, or at least 1.5 ppm (e.g. from about 1 ppm to about 4 ppm, such as about 1.25 ppm to about 3.5 ppm, or about 1.5 ppm to about 3 ppm). In other configurations, the concentration of volatile conjugated aldehyde containing compounds may be even higher, e.g. at least 1.1 ppm, at least 1.4 ppm, or at least 2 ppm. A skilled person will appreciate that a higher proportion of conjugated aldehyde in the volatile composition will lead to a higher proportion of conjugated aldehyde in the headspace.

Such methods have been found to be effective in counteracting malodor in a waste bin. A total concentration of malodor-causing amines and thiols in a home waste bin is almost always less than 3 ppm, and is generally substantially lower than this level. Accordingly, a concentration of volatile aldehyde containing compounds around or above this level is able to effectively counteract malodor in the waste bin headspace. The concentration of volatile aldehyde containing compounds within a headspace of the waste bin may be determined by activating an air freshening product, and leaving the air freshening product adhered to an interior surface of a waste bin having a volume of 50 L for a time period of 12 hours at 22° C., atmospheric pressure and a relative humidity of 50% (e.g. for a time period of 6 hours or 1 hour), taking a sample of the headspace and subjecting the sample to gas chromatography analysis. After being left to equilibrise overnight, the devices tested in the below Examples were able to provide a concentration of volatile aldehyde containing compounds of about 3.2 ppm within 15 minutes from opening and closing a waste bin lid, based on analysis of VOC content in the waste bin headspace. This level was achieved using a freshening composition containing 28.5 wt. % of volatile aldehyde-containing compounds, where the volatile aldehyde-containing compounds had a weight average vapor pressure of 0.55 Torr at 25° C., and the freshening composition had a weight average vapor pressure of 0.41 Torr at 25° C. In this method, the air freshening product and freshening composition may have any additional feature or combination of features described herein. In some configurations, the mixture of volatile aldehyde-containing compounds may comprise at least 8 wt. %, by weight of the freshening composition, of volatile aldehyde-containing compounds having a conjugated aldehyde group. The mixture of volatile aldehyde-containing compounds may have any feature or combination of features described hereinabove.

Also provided herein is an air freshening product that provides a scaled magnitude estimation score as assessed by Sensory Malodor Test Method 2 of less than or equal to 0.5 for each of 1 hour, 2 weeks, 4 weeks and 6 weeks, wherein the assessment is conducted using a waste bin having an internal volume of 50 L, at a temperature of 22° C., a relative humidity of 50%, and atmospheric pressure. In some configurations the scaled magnitude estimation score is the Open Bin scaled magnitude estimation score. In some configurations, the Open Bin scaled magnitude estimation score is less than or equal to 0.4 for each of 1 hour, 2 weeks, 4 weeks and 6 weeks. In some configurations the scaled magnitude estimation score is the Remove Liner scaled magnitude estimation score, and the Remove Liner scaled magnitude estimation score is less than or equal to 0.5 for 1 hour, and less than or equal to 0.4 for each of 2 weeks, 4 weeks and 6 weeks. A scaled magnitude estimation score of less than 0.5 corresponds to a reduction in malodor, as assessed by the panelists, of at least 2×. Therefore, in some configurations, the air freshening product may provide a perceived reduction of malodor of at least 2×, as assessed by Sensory Malodor Test Method 2. The air freshening product may comprise a freshening composition as described herein. In particular, the air freshening product may comprise a freshening composition that comprises at least 10 wt. %, based on the weight of the freshening composition, of a mixture of volatile aldehyde-containing compounds, the volatile aldehyde-containing compounds each having a vapor pressure of at least 0.018 Torr at 25° C. In some configurations, the freshening composition may comprise at least 8 wt. %, by weight of the freshening composition, of volatile aldehyde-containing compounds having a conjugated aldehyde group. In some configurations, the air freshening product may comprise a housing and a cartridge containing a freshening composition, where the freshening composition is configured to volatilise from the cartridge. The air freshening product may have any additional feature or combination of features described herein.

Although the freshening composition of the invention is generally described above in the context of a liquid composition for volatilisation from a membrane-containing air freshening product, the freshening composition of the invention may be provided in any appropriate form, such as part of, incorporated into, or absorbed/adsorbed into, a spray, aerosol, polymer gel, tablet, fibrous product, woven or non-woven sheet, or combination thereof. Suitable such product forms are known to a person skilled in the art.

Vapor pressures for specific compounds not defined herein may be obtained from the Percepta Batch Module, version 14.50, available from Advanced Chemistry Development/ACD Labs, using the PhysChem Suite tool.

The invention is illustrated by the following Examples, which are not to be construed as limiting the scope of the appended claims.

### EXAMPLES

### General Apparatus and Methods

The following apparatus was used for the evaluation of malodor and perfume intensity.
1. Balance (Scale: Ohaus AA210 S/N 11131122540) or equivalent.
2. Volatile composition cartridges containing 5 ml of perfume composition, as described in the Figures of U.S. provisional patent application No. 63/677,126 (published as part of the file wrapper of priority-claiming applications), and its corresponding non-provisional patent application no. U.S. Ser. No. 19/282,107.
3. Housing to hold the cartridges as described in the Figures of U.S. provisional patent application No. 63/677,126 (published as part of the file wrapper of priority-claiming applications), and its corresponding non-provisional patent application no. U.S. Ser. No. 19/282,107.
4. 3M Scotch Weld Applicator TC and glue, #3797-TC or equivalent.
5. Electric skillet (Presto or equivalent).
6. PCW (post-consumer waste)-50 fabric.
7. 114 L waste bin with carousel hanger.
8. 50 L waste bin with a lid (Ikea Knockla pedal bin or equivalent) and bin bag (Glad kitchen bin bag or equivalent).
9. Evaluation room to accommodate waste bin with the following measurements, air flow, temperature and relative humidity or equivalent:
   a. Room Dimensions: 1.6 m long×1.1 m wide×2.12 m high or 3.73 m³
   b. Air flow: 2 air exchanges per hour during evaluation, 8 air exchanges per hour during breaks between different panelists.
   c. Temperature and % Relative Humidity
      i. Average Temperature: 22° C.±2° C.
      ii. Average % Relative Humidity: 50%±5%

The procedure to prepare scent cartridges is as follows:
1. Load a cartridge with the volatile composition in such a way as to provide a sealed cartridge where the membrane is not yet wetted. For instance, one may pierce a volatile composition cartridge by cutting in it a hole that allows for insertion of an 18-gauge needle.
2. Fill the cartridge with 5 ml of perfume. This is equivalent to 4750 mg of the standard perfume. The volume may need to be adjusted based on the density of the composition of interest.
3. Seal the insertion hole with hot melt adhesive.
4. Measure and record the weight of the device.
5. Insert the cartridge into a housing for holding and orienting the cartridge, and ensure that the cartridge is set correctly within the housing to ensure proper air flow therethrough.
6. Activate the cartridge by any appropriate means to allow contact between the perfume and the membrane, thereby wetting the membrane. In the examples disclosed herein this activation was achieved by sliding the cartridge into the housing, which breaks a breakable seal between the perfume and the membrane.

### Malodor Cocktail

The malodor cocktail used in the test methods/Examples below was obtained as follows.
1. Dried fish:
   Place one piece of dried fish of desired weight as specified in test method on petri dish. Dried fish source is “Dried Sole Fish (Happy Family) packed by CMM Marketing Management Pte Ltd” or equivalent.
2. Bacon grease:
   Hang 4 PCW fabrics from carousel hanger of the 114 L waste bin.
   Using spatula, place 8 ounces of bacon grease in the skillet and cover with skillet lid. Run electric cord through 1.5″ hole in the waste bin. Set skillet to 250° F. Allow 15 minutes for equilibration. Remove skillet lid.
   Place lid on the waste bin and begin timing.
   Turn the carousel in the lid, manually, at approximately 15 RPM for 3 minutes.
   Remove the fabrics from the carousel hanger.
   Cut 1 piece of fabric to desired size as specified in test method and place on a Petri dish.
3. Cooked Garlic:
   Weigh out 80 g of cooking oil and place in skillet and cover with skillet lid. Plug in skillet and turn skillet to 220° F. and allow 10 minutes for equilibration.
   Measure 50 g of commercially prepared minced garlic in water (Spice World of Polaner or equivalent) and place in hot oil in skillet.
   Place lid onto skillet.
   Cook for 2½ minutes. Stir at the halfway mark with spatula. All garlic should be translucent, with a portion starting to brown but not burn.
   Remove the garlic and weigh desired amount as specified in test method into a Petri dish.
4. Broccoli:
   Purchase fresh whole broccoli and keep in a fridge for 5 days.
   Extract desired amount as specified in test method of the broccoli florets and place on a petri dish.
5. Bathroom:
   A synthetic bathroom malodor mixture was obtained by mixing 6.7 ml of a commercially available synthetic urine malodor mixture and 20 ml of a commercially available synthetic fecal malodor mixture.
   Add desired volume of bathroom malodor as specified in test method to a petri dish.

For each of Malodor Test Method 1 and 2 below, the amount of malodor cocktail used was chosen to represent a realistic level of malodor in a kitchen bin. Malodor Test Method 1 was used with multiple trained sensory panelists, and used a higher malodor level to investigate the ability of the invention to counteract malodor in a very smelly kitchen bin. Malodor Test Method 2 was used with an expert sensory panelist and perfumier, so a lower level of malodor was used in order to utilize the expert panelist's ability to detect subtle differences in malodor.

### Sensory Malodor Test Method 1

1. Prepare scent cartridge, e.g. as detailed above.
2. Place bin liner onto separate waste bins.
3. Stick device (i.e., housing containing volatile composition cartridge) on the waste bin lid 24 hours before evaluation.
4. Place waste bin in evaluation room.
5. Prepare malodor cocktail as detailed above with the following amounts:
   a. Dried fish: 0.03 g
   b. Bacon grease: 25 cm² of fabric
   c. Cooked garlic: 0.08 g
6. Place the malodor cocktail in the waste bin 1 hour before evaluation.
7. Arrange expert panelist/perfumier for the evaluation.
8. For each panelist/perfumier,
   a. Enter the evaluation room.
   b. Step on the foot pedal to open the waste bin.
   c. Evaluate smell for 5 seconds.
   d. Remove feet from foot pedal and ensure waste bin is closed.
   e. Provide the perfume and malodor intensity scores.
   f Exit the evaluation room.
   g. Take a 2-minute break before next evaluation.
   h. Repeat steps a to g for all the waste bins.
   i. Re-enter the first evaluation room.
   j. Step on the foot pedal to open the waste bin.
   k. Remove the liner from the waste bin.
   l. Remove feet from foot pedal and ensure waste bin is closed.
   m. Liner should be placed at hip level of panelist with arms kept straight.
   n. Evaluate smell for 5 seconds.
   o. Provide the perfume and malodor intensity scores.
   p. Place the liner back into the bin.
   q. Exit the evaluation room.
   r. Take a 2-minute break before next evaluation.
   s. Repeat steps i to r for all the waste bins.
9. For the panelist, follow the steps outlined in step 8 of test 1.

For Sensory Malodor Test Method 1, the malodor intensity was scored as follows.
0: No malodor present
10: I think there is malodor present (unsure)
20: I detect something, but can I recognize it?
25: Slight malodor present
40: Slight to moderate malodor present
50: Moderate malodor present
60: Moderate to strong malodor present
75: Strong malodor present
100: Extremely strong malodor present

### Sensory Malodor Test Method 2

This method is adapted from ASTM E1697.
1. Prepare scent cartridge, e.g. as detailed above.
2. Place bin liner onto separate waste bins.
3. Stick device (i.e., housing containing volatile composition cartridge) on the waste bin lid 24 hours before evaluation.
4. Place waste bin in evaluation room.
5. Prepare malodor cocktail as detailed above with the following amounts:
   a. Dried fish: 0.12 g
   b. Bacon grease: 100 cm² of fabric
   c. Cooked garlic: 0.32 g
   d. Broccoli: 15 g
   e. Bathroom: 20 μL
6. Place the malodor cocktail in the waste bin with device 1 hour before evaluation.
7. Place the malodor cocktail in another 3 separate waste bins without device 1 hour before evaluation.
8. Arrange 14 trained sensory panelists/perfumiers for the evaluation.
9. For each panelist/perfumier,
   a. Enter the first evaluation room (no device in waste bin).
   b. Step on the foot pedal to open the waste bin.
   c. Evaluate smell for 5 seconds.
   d. Remove feet from foot pedal and ensure waste bin is closed.
   e. Exit the evaluation room.
   f. Take a 2-minute break before next evaluation.
   g. Enter the second evaluation room (no device in waste bin) and repeat steps b to f.
   h. Provide the magnitude estimation score (ASTM E1697) for second room vs. first room.
   i. Repeat steps a to h for third (no device in waste bin) and fourth (device in waste bin) evaluation rooms.
   j. Re-enter the first evaluation room.
   k. Step on the foot pedal to open the waste bin.
   l. Remove the liner from the waste bin.
   m. Remove feet from foot pedal and ensure waste bin is closed.
   n. Liner should be placed at hip level of panelist with arms kept straight.
   o. Evaluate smell for 5 seconds.
   p. Place the liner back into the bin.
   q. Exit the evaluation room.
   r. Take a 2-minute break before next evaluation.
   s. Enter the second evaluation room and repeat steps k to r.
   t. Provide the magnitude estimation score (ASTM E1697) for second room vs. first room.
   u. Repeat steps j to t for third (no device in waste bin) and fourth (device in waste bin) evaluation rooms.
10. Repeat step 9 for all the panelists, with a time gap of 15 minutes between the start of each panelist's evaluation.
11. Calculate the average scaled magnitude estimation score based on ASTM E1697. 12. For the waste bin with device, continue using the device for 2 more weeks with opening of waste bin every day for 25 times and switching to new malodor twice every week.
13. Repeat steps 7 to 11 to obtain data for device at 1 hour, 2 weeks, 4 weeks and 6 weeks timepoints.

### Analytical Malodor Test Method 1

1. Prepare scent cartridge as detailed above.
2. Place bin liner onto separate waste bins.
3. Stick device (i.e., housing containing volatile composition cartridge) on the waste bin lid 24 hours before evaluation.
4. Place waste bin in evaluation room.
5. Prepare malodor cocktail as detailed above with the following amounts:
   a. Dried fish: 0.12 g
   b. Bacon grease: 100 cm² of fabric
   c. Cooked garlic: 0.32 g
   d. Broccoli: 15 g
   e. Bathroom: 20 μL
6. Place the malodor cocktail in the waste bin with device.
7. Place the malodor cocktail in another separate waste bin without device.
8. Place 3 gas collection pumps in each of the waste bins. The gas pumps are set to collect the headspace after either 30 minutes, 3 hours or 24 hours.
9. Send the headspace collected for GC analysis to calculate the amount of different chemicals at different timepoints.
10. Calculate the “% change vs. 30 minutes” metric using below equation
   a. (Amount of chemical at 3 or 24 hours−Amount of chemical at 30 minutes)*100%
      i. 3 hours timepoint for diallyl disulfide and allyl 1-propenyl disulfide
      ii. 24 hours timepoint for trimethylamine, 2-propenoic acid, 3-penten-2-one, dimethyl disulfide and diallyl trisulfide
11. For the waste bin with device, continue using the device for 2 more weeks with opening of waste bin every day for 25 times and switching to new malodor twice every week.
12. Repeat steps 6 to 10 to obtain data for device at 1 hour, 2 weeks, 4 weeks and 6 weeks timepoints.

### Analytical Malodor Test Method 2

Each of the malodorous gases was tested in a different 50 L gas bag.
1. Prepare scent cartridge as detailed above.
2. Place housing containing volatile composition cartridge in a 50 L gas bag with predetermined number of sheets of dry or wet kitchen towel. Wet kitchen towel can be obtained by spraying dry towel with water from spray bottle (2 full compressions).
   1× dry paper towel for butylamine, pentylamine
   5× dry paper towel for ammonia, dimethyl disulfide, trimethylamine, hydrogen sulfide
   5× wet paper towel for butanethiol
3. Leave device to dwell inside bag for 1 hour.
4. Fill the bag with malodorous gas at the prescribed concentration and begin sample collection (this is time=0 hr).
   1 ppm for trimethylamine, butanethiol, hydrogen sulfide
   2 ppm for dimethyl disulfide
   5 ppm for butylamine, pentylamine
   30 ppm for ammonia
5. In addition, prepare another 50 L bag containing the predetermined number of dry or wet kitchen towel and inject with malodorous gas. This will be used as a control (without composition according to the invention).
6. Measure gas concentration with a detector tube at predetermined time from the time when malodorous gas is filled.
   30 minutes for butylamine, pentylamine
   2 hours for trimethylamine
   4 hours for dimethyl disulfide
   24 hours for butanethiol, ammonia, hydrogen sulfide
3. Calculate the “% better elimination vs. control” metric using below equation
   ((Amount of gas at start for product−Amount of gas at end for product)/(Amount of gas at start for control−Amount of gas at end for control))−1)*100%

### Example 1: Sensory Evaluation of Effect of Total Aldehyde Level

Freshening compositions comprising varying levels of volatile aldehyde-containing compounds were tested for their ability to counteract malodor, based on Sensory Malodor Test Method 1.

A mixture of volatile aldehyde-containing compounds was prepared as listed in Table 4 below. The mixture had a weight average vapor pressure of 1.64 Torr at 25° C.

TABLE 4

| | | Weight | Vapor pressure |
|---|---|---|---|
| CAS | Aldehyde | % | at 25° C. (Torr) |
| 6728-26-3 | Trans-2-hexenal | 0.88 | 4.62 |
| 100-52-7 | Benzaldehyde | 5.90 | 0.974 |
| 55722-59-3 | 3,7-dimethylocta-2,6-dienal | 44.2 | 0.0712 |
| | (Citral | | |
| 104-55-2 | (E)-3-phenylprop-2-enal | 3.5 | 0.0265 |
| | (Cinnamaldehyde) | | |
| | Non-conjugated aldehydes | 45.52 | |

Four test compositions were prepared as listed in Table 5 below. The compositions comprised 5, 15, 30 and 50 wt. 0% of the aldehyde mixture in Table 4, respectively. The balance of the compositions was composed of a mixture of solvents, containing 21.4 wt. % 3-methoxy-3-methyl-1-butanol (MMVB), 42.85 wt. 00 dimethyl adipate (DMA), and 35.75 wt. 00 dipropylene glycol monomethyl ether (DPGMVE).

TABLE 5

| | | Conjugated | |
|---|---|---|---|
| | Total Aldehyde | Aldehyde | Solvent |
| Composition | (wt. %) | (wt. %) | (wt. %) |
| A1 (Comparative) | 5 | 2.72 | 95 |
| B1 (Inventive) | 15 | 8.17 | 85 |
| C1 (Inventive) | 30 | 16.34 | 70 |
| D1 (Inventive) | 50 | 27.2 | 50 |

The ability of the four compositions to counteract malodor in a waste bin was tested according to Sensory Malodor Test Method 1, and the results are set out in Table 6 below.

TABLE 6

| | | Control | Comparative | Inventive | Inventive | Inventive |
|---|---|---|---|---|---|---|
| | | Malodor | Malodor + | Malodor + | Malodor + | Malodor + |
| | | only | Mixture A1 | Mixture B1 | Mixture C1 | Mixture D1 |
| Waste | Perfume | — | 25 | 30 | 30 | 40 |
| bin | Intensity | | | | | |
| Opening | Malodor | 40 | 35 | 30 | 25 | 15 |
| | Intensity | | | | | |
| | Scent | Strong | Some citrus | Malodor | Malodor | Unclear |
| | Character | oily, | scent, much | still present | still | malodor |
| | | fishy | more | | present | |
| | | | malodor | | | |
| Remove | Perfume | — | 20 | 25 | 35 | 40 |
| Liner | Intensity | | | | | |
| | Malodor | 45 | 45 | 35 | 20 | 10 |
| | Intensity | | | | | |
| | Scent | Quite | Mainly | Less oily, | Malodor | Almost no |
| | Character | garlic, | malodor, | garlic, fatty | becomes | perception |
| | | fishy | unclear | malodor | fainter vs | of malodor, |
| | | | perfume | | Mixture B | mostly |
| | | | | | | aldehyde |
| | | | | | | perfume |
| | | | | | | odor |

The results demonstrate the ability of compositions according to the invention to counteract malodor in a waste bin, improving a consumer's experience when opening the waste bin and removing a waste bin liner from the waste bin.

### Example 2: Sensory Evaluation of Effect of Conjugated Aldehyde Level

To investigate the effect of conjugated aldehydes on counteracting malodor, freshening compositions containing the same total amount of aldehyde, but with differing amounts of conjugated aldehyde were prepared.

Mixtures of conjugated and non-conjugated aldehydes were prepared as set out in Tables 7 and 8 below. To confirm that differences in effect were provided by the conjugation of the aldehyde, the amount of each aldehyde was adjusted to keep a consistent weight average vapor pressure for each mixture. The specific aldehydes used in each mixture were selected to provide mixtures having similar smell profiles with equal weight average vapor pressures, whilst also using aldehydes that are known to be useful in perfumery. The invention is not limited to the specific aldehydes listed below, and a skilled person will appreciate that equivalent results may be obtained with mixtures of different aldehydes.

TABLE 7
Conjugated aldehyde mixture

| | | | Vapor Pressure |
|---|---|---|---|
| CAS | Conjugated Aldehyde | Wt. % | at 25° C. (Torr) |
| 6728-26-3 | Trans-2-hexenal | 18.5 | 4.62 |
| 100-52-7 | Benzaldehyde | 18.5 | 0.974 |
| 104-55-2 | (E)-3-phenylprop-2-enal | 31.5 | 0.0265 |
| | (Cinnamaldehyde) | | |
| 123-11-5 | 4-methoxybenzaldehyde | 31.5 | 0.0249 |
| | (Anisic aldehyde) | | |

TABLE 8
Non-conjugated aldehyde mixture

| | Non-conjugated | | Vapor Pressure |
|---|---|---|---|
| CAS | Aldehyde | Wt. % | at 25° C. (Torr) |
| 124-13-0 | Octyl aldehyde | 50 | 2.07 |
| 33885-51-7 | Pino Acetaldehyde | 25 | 0.0304 |
| 7775-00-0 | Cyclemax | 25 | 0.0182 |

Four test compositions A2-D2 were prepared, as set out in Table 9. All of the compositions A2-D2 contained 30 wt. % of volatile aldehydes, though with varying amounts of conjugated aldehydes. All of compositions A2-D2 contained 70 wt. % solvent, which consisted of a mixture of 21.4 wt. % 3-methoxy-3-methyl-1-butanol (MMB), 42.85 wt. % dimethyl adipate (DMA), and 35.75 wt. % dipropylene glycol monomethyl ether (DPGME).

TABLE 9

| | Conjugated | Non-conjugated | |
|---|---|---|---|
| | Aldehyde | Aldehyde | |
| | Mixture of | Mixture of | |
| | Table 6 | Table 7 | Solvent |
| Composition | (wt. %) | (wt. %) | (wt. %) |
| A2 | 0 | 30 | 70 |
| B2 | 10 | 20 | 70 |
| C2 | 20 | 10 | 70 |
| D2 | 30 | 0 | 70 |

The compositions A2-D2 were tested according to Sensory Malodor Test Method 1. Results are listed in Table 10 below.

TABLE 10

| | | Control | Comparative | Inventive | Inventive | Inventive |
|---|---|---|---|---|---|---|
| | | Malodor | Malodor + | Malodor + | Malodor + | Malodor + |
| | | only | A2 | B2 | C2 | D2 |
| Waste | Perfume | — | 40 | 45 | 45 | 47.5 |
| bin | Intensity | | | | | |
| Opening | Malodor | 40 | 25 | 15 | 10 | 0 |
| | Intensity | | | | | |
| | Scent | Strong | Some | Unclear | Almost no | No |
| | Character | oily, fishy | dirty/fishy | malodor | malodor | evidence of |
| | | | odor | | | malodor |
| Remove | Perfume | — | 42.5 | 42.5 | 45 | 42.5 |
| Liner | Intensity | | | | | |
| | Malodor | 45 | 30 | 22.5 | 20 | 10 |
| | Intensity | | | | | |
| | Scent | Quite | Oily garlicy | Not garlic | Slightly | Even less |
| | Character | garlic, | bag odor | like. Some | oily | oily vs |
| | | fishy | | oily | malodor | previous |
| | | | | malodor | | conjugated |
| | | | | | | levels |

As is clear from Table 10, the ability of the compositions to counteract malodor increases with increased level of conjugated aldehyde. Despite having similar scents, the same total level of aldehyde, and the same weight average vapor pressure, composition D2 was able to fully eliminate malodor, while composition A2 still resulted in a noticeable malodor. This indicates that conjugated aldehydes are better able to counteract malodor than non-conjugated aldehydes.

### Example 3: Analytical evaluation

To confirm the validity of sensory experiments, analytical test methods were used to demonstrate the correlation between reduced sensory malodor scores, and reduced levels of malodor gases.

The ability of a composition according to the invention (composition A3) to reduce malodor was confirmed according to Analytical Malodor Test Method 1, Analytical Malodor Test Method 2, and Sensory Malodor Test Method 2.

The first tested composition A3 comprises major aldehyde components listed in Table 11. Composition A3 had a citrus aroma and a weight average vapor pressure of 0.426 Torr at 25° C. The weight average vapor pressure of the aldehyde component was 0.50 Torr at 25° C.

TABLE 11
Major aldehyde components of composition A3

| | | Conjugated | |
|---|---|---|---|
| | | or non- | |
| CAS | Aldehyde | conjugated | Weight % |
| 55722-59-3 | 3,7-dimethylocta-2,6- | Conjugated | 16.30 |
| | dienal (Citral) | | |
| 100-52-7 | Benzaldehyde | Conjugated | 3.91 |
| 104-55-2 | (E)-3-phenylprop-2- | Conjugated | 2.34 |
| | enal (Cinnamaldehyde) | | |
| 112-31-2 | Decanal | Non-conjugated | 2.29 |
| 124-19-6 | Nonanal | Non-conjugated | 2.05 |
| 124-13-0 | Octanal | Non-conjugated | 1.49 |
| | Other conjugated | Conjugated | 0.25 |
| | aldehydes | | |
| | Other non-conjugated | Non-conjugated | 3.23 |
| | aldehydes | | |
| | (<1 wt. % each) | | |
| | | Solvents | 15 |
| | | Perfume raw | Balance |
| | | materials | to 100 |
| | | Total aldehyde | 31.86 |
| | | Total conjugated | 22.80 |
| | | aldehyde | |

### Waste Bin Headspace Analysis Analytical Malodor Test Method 1)

As shown in Table 12 below, composition A3 was able to significantly reduce the presence of several malodor-causing compounds in the headspace of waste bin containing a malodor cocktail. This effect lasted for at least 6 weeks. Positive values indicate an increase in the level of the malodor-causing compound in the headspace, while negative values indicate a decrease.

TABLE 12

| | | Malodor Only | | | | Malodor + composition A3 | | | |
|---|---|---|---|---|---|---|---|---|---|
| | | (% change vs. 30 mins) | | | | (% change vs. 30 mins) | | | |
| Malodor-causing | | | At 2 | At 4 | At 6 | | At 2 | At 4 | At 6 |
| compound | T1 | At T1 | weeks | weeks | weeks | At T1 | weeks | weeks | weeks |
| Trimethylamine | 24 hrs | −2% | −1% | −4% | −5% | −82% | −77% | −55% | −27% |
| 2-propenoic acid | 24 hrs | +74% | +101% | +70% | +216% | +31% | −11% | −40% | +33% |
| 3-penten-2-one | 24 hrs | +202% | +62% | +39% | +116% | −65% | −15% | −70% | −81% |
| Dimethyl disulfide | 24 hrs | +130% | +156% | +94% | −7% | +16% | −98% | −51% | −86% |
| Diallyl disulfide | 3 hrs | +80% | −4% | +32% | +15% | −75% | −78% | −21% | −36% |
| Diallyl trisulfide | 24 hrs | +403% | +8% | +150% | +140% | +13% | −57% | +64% | +25% |
| Allyl 1-propenyl | 3 hrs | +97% | −17% | +52% | +19% | −94% | −98% | −89% | −70% |
| disulfide | | | | | | | | | |

### Gas Bag Analysis (Analytical Malodor Test Method 2)

As shown in Table 13, composition A3 was able to provide a far greater decrease in the level of tested malodor-causing compounds as compared to a control representing the natural dissipation rate.

TABLE 13

| | Malodor-causing | % better elimination |
|---|---|---|
| Malodor Type | compound | vs. malodor only |
| N-containing (raw fish, | Pentylamine | 600 |
| rotten meat, diapers) | Butylamine | 325 |
| | Ammonia | 173 |
| | Trimethylamine | 100 |
| S-containing (rotten | Butanethiol | 400 |
| vegetables, raw meat, | Hydrogen sulfide | 100 |
| rotten eggs, diapers) | Dimethyl disulfide | 300 |

### Further Sensory Analysis (Sensory Malodor Test Method 2)

The scaled magnitude estimation score is an indicator of the weakness of the malodor as compared to a control. The score is an average from an assessment by 14 trained sensory panelists. A score of 1 indicates the same malodor intensity, a score of 0.5 indicates 2× weaker malodor, a score of 0.25 indicates 4× weaker malodor, and a score of 0.2 indicates 5× weaker malodor.

As shown in Table 14, composition A3 provided a large decrease in malodor as compared to a control. This decrease in malodor was evident after only 1 hour, and lasted for at least six weeks.

TABLE 14
Results for composition A3

| | | Average Scaled |
|---|---|---|
| Touch | | Magnitude |
| point | Timepoint | Estimation Score |
| Open Bin | After 1 hour | 0.38 |
| | After 2 weeks natural aging | 0.24 |
| | After 4 weeks natural aging | 0.22 |
| | After 6 weeks natural aging | 0.20 |
| Remove | After 1 hour | 0.46 |
| Liner | After 2 weeks natural aging | 0.31 |
| | After 4 weeks natural aging | 0.23 |
| | After 6 weeks natural aging | 0.28 |

The results in Tables 12-14 confirm that the reduction in malodor as assessed by sensory panelists (Table 14) is consistent with reduction in malodor-causing compounds as assessed by analytical techniques (Tables 12 and 13). These results therefore validate the accuracy of sensory experimental results in Examples 1 and 2.

A further composition B3 according to the invention was also assessed using Sensory Malodor Test Method 2, with assessment conducted by 14 trained sensory panelists. The major aldehyde components of composition B3 are listed in Table 15, and results are provided in Table 16 below.

TABLE 15
Major aldehyde components of composition B3

| | | Conjugated | |
|---|---|---|---|
| | | or non- | |
| CAS | Aldehyde | conjugated | Weight % |
| 55722-59-3 | 3,7-dimethylocta-2,6- | Conjugated | 8.51 |
| | dienal (Citral) | | |
| 123-11-5 | 4-methoxybenzaldehyde | Conjugated | 2.49 |
| | (Anisic aldehyde) | | |
| 112-31-2 | Decanal | Non-conjugated | 2.33 |
| 68039-49-6 | Triplal ® | Non-conjugated | 2.08 |
| 100-52-7 | Benzaldehyde | Conjugated | 1.83 |
| 124-13-0 | Octanal | Non-conjugated | 1.24 |
| 104-55-2 | (E)-3-phenylprop-2- | Conjugated | 1.09 |
| | enal (Cinnamaldehyde) | | |
| 124-19-6 | Nonanal | Non-conjugated | 0.95 |
| 6784-13-0 | 3-(4-methyl-1-cyclohex- | Non-conjugated | 0.83 |
| | 3-enyl)butanal | | |
| | Other conjugated | Conjugated | 0.15 |
| | aldehydes | | |
| | Other non-conjugated | Non-conjugated | 2.58 |
| | aldehydes | | |
| | (<0.6 wt. % each) | | |
| | | Solvents | 12 |
| | | Perfume raw | Balance |
| | | materials | to 100 |
| | | Total aldehyde | 24.08 |
| | | Total conjugated | 14.07 |
| | | aldehyde | |

TABLE 16
Results for composition B3

| | | Average Scaled |
|---|---|---|
| | | Magnitude |
| Touch point | Timepoint | Estimation Score |
| Open Bin | After 1 hour | 0.16 |
| | After 2 weeks natural aging | 0.22 |
| | After 4 weeks natural aging | 0.19 |
| | After 6 weeks natural aging | 0.25 |
| Remove Liner | After 1 hour | 0.20 |
| | After 2 weeks natural aging | 0.33 |
| | After 4 weeks natural aging | 0.34 |
| | After 6 weeks natural aging | 0.36 |

As for composition A3, composition B3 was also able to significantly reduce malodor experienced by a consumer, both when opening a waste bin and removing the liner from the waste bin.

The results in Examples 1 to 3 confirm that compositions according to the invention, which contain significant amounts of conjugated aldehydes, are able to effectively reduce malodor in a wase bin. Furthermore, the results demonstrate that conjugated aldehydes are able to better reduce malodor in a waste bin than non-conjugated aldehydes. The reduction in malodor experienced by a consumer when removing the liner from the waste bin confirms that the compositions of the invention are able to reduce malodor both in the air, and also reduce malodor on surfaces (e.g., malodor adsorbed or absorbed to the bin liner and other porous surfaces within the waste bin).

As used in this specification and the claims that follow, the articles “a”, “an”, and “the” include singular and plural references unless the context clearly dictates otherwise. As such, the terms “a” or “an”, “one or more” and “at least one” can be used interchangeably herein. Thus, for example, “a component” may include one or more components unless the reference is specifically indicated as being singular.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A freshening composition comprising:
from about 0.01 wt. % to about 90 wt. %, by weight of the freshening composition, of one or more non-aldehyde perfume raw materials; and
from about 10 wt. % to about 70 wt. %, by weight of the freshening composition, of a mixture of volatile aldehyde-containing compounds, the volatile aldehyde-containing compounds each having a vapor pressure of at least 0.018 Torr at 25° C.;
wherein:
the mixture of volatile aldehyde-containing compounds comprises at least 8 wt. %, by weight of the freshening composition, of volatile aldehyde-containing compounds having a conjugated aldehyde group.

2. The freshening composition according to claim 1, wherein the freshening composition comprises from about 12 wt. % to about 50 wt. %, by weight of the freshening composition, of the mixture of volatile aldehyde-containing compounds.

3. The freshening composition according to claim 1, wherein the freshening composition comprises from about 22 wt. % to about 40 wt. %, by weight of the freshening composition, of the mixture of volatile aldehyde-containing compounds.

4. The freshening composition according to claim 1, wherein the mixture of volatile aldehyde-containing compounds has a weight average vapor pressure of from about 0.33 Torr to about 0.6 Torr at 25° C.

5. The freshening composition according to claim 1, wherein the mixture of volatile aldehyde-containing compounds comprises at least 30%, by weight of the mixture of volatile aldehyde-containing compounds, of volatile aldehyde-containing compounds having a vapor pressure of greater than 0.1 Torr at 25° C.

6. The freshening composition according to claim 1, wherein the mixture of volatile aldehyde-containing compounds comprises at least 40%, by weight of the mixture of volatile aldehyde-containing compounds, of volatile aldehyde-containing compounds having a vapor pressure of greater than 0.1 Torr at 25° C.

7. The freshening composition according to claim 1, wherein the mixture of volatile aldehyde-containing compounds comprises at least 10%, by weight of the freshening composition, of volatile aldehyde-containing compounds having a conjugated aldehyde group.

8. The freshening composition according to claim 1, wherein:
the freshening composition comprises at least 12%, by weight of the freshening composition, of the mixture of volatile aldehyde-containing compounds; and
wherein the mixture of volatile aldehyde-containing compounds comprises at least 12%, by weight of the freshening composition, of volatile aldehyde-containing compounds having a conjugated aldehyde group.

9. The freshening composition according to claim 1, wherein a weight ratio of conjugated aldehyde-containing compounds to non-conjugated aldehyde-containing compounds in the mixture of volatile aldehyde-containing compounds is at least 1.05.

10. The freshening composition according to claim 9, wherein a weight ratio of conjugated aldehyde-containing compounds to non-conjugated aldehyde-containing compounds in the mixture of volatile aldehyde-containing compounds is from 1.1 to 5.

11. The freshening composition according to claim 1, wherein the mixture of volatile aldehyde-containing compounds comprises at least 10 wt. %, based on the amount of the mixture of volatile aldehyde-containing compounds, of cinnamaldehyde, benzaldehyde, or a mixture thereof.

12. The freshening composition according to claim 1, wherein the freshening composition has a weight average vapor pressure of from 0.37 to 0.6 Torr at 25° C.

13. The freshening composition according to claim 12, wherein the freshening composition has a weight average vapor pressure of from 0.4 to 0.45 Torr at 25° C.

14. The freshening composition according to claim 1, wherein the one or more non-aldehyde perfume raw materials comprise at least 4 wt. %, by weight of the freshening composition, of non-aldehyde perfume raw materials having a citrus aroma.

15. A freshening composition comprising:
from about 0.01 wt. % to about 90 wt. %, by weight of the freshening composition, of one or more non-aldehyde perfume raw materials; and
from about 10 wt. % to about 70 wt. %, by weight of the freshening composition, of a mixture of volatile aldehyde-containing compounds, the volatile aldehyde-containing compounds each having a vapor pressure of at least 0.018 Torr at 25° C.;
wherein:
a weight ratio of conjugated aldehydes to non-conjugated aldehydes in the mixture of volatile aldehyde-containing compounds is at least 1.85:1.

16. A freshening composition comprising:
from about 0.01 wt. % to about 90 wt. %, by weight of the freshening composition, of one or more non-aldehyde perfume raw materials; and
from about 10 wt. % to about 70 wt. %, by weight of the freshening composition, of a mixture of volatile aldehyde-containing compounds, the volatile aldehyde-containing compounds each having a vapor pressure of at least 0.018 Torr at 25° C.;
wherein:
the mixture of volatile aldehyde-containing compounds comprises at least 5%, by weight of the freshening composition, of volatile aldehyde-containing compounds having a conjugated aldehyde group; and
the mixture of volatile aldehyde-containing compounds comprises at least 5%, by weight of the freshening composition, of volatile aldehyde-containing compounds having a vapor pressure of at least 0.1 Torr at 25° C.

17. An air freshening product comprising:
a reservoir enclosed by a membrane, the reservoir containing the freshening composition according to claim 1, where the freshening composition is configured to evaporate through or from the membrane.

18. The air freshening product of claim 17, wherein the air freshening product requires an activation step to enable evaporation of the freshening composition through or from the membrane.

19. A method of reducing malodor in a waste bin, comprising:
placing an air freshening product according to claim 17 into or adjacent to the waste bin; and
allowing the freshening composition to evaporate from the air freshening product.
